(19) United States
(12) Patent Application Publication
(10) Pub. No.: US 2006/0064129 A1
(43) Pub. Date: Mar. 23, 2006

(54) Device for electric stimulation of the lymphatic system and applications of such device

(76) Inventor: Laurent Pujol, Cergy Pontoise Cedex (FR)

Correspondence Address: ARENT FOX PLLC, 1050 CONNECTICUT AVENUE, N.W., SUITE 400, WASHINGTON, DC, 20036, US

(21) Appl. No.: 11/271,912

(22) Filed: Nov. 14, 2005

Related U.S. Application Data
(63) Continuation of application No. 10/069,125, filed on Jun. 20, 2002, now abandoned.

(30) Foreign Application Priority Data
Sep. 1, 1999 (FR) 99/11043

(51) Int. Cl. A61N 1/00

(52) U.S. Cl. 607/2

(57) ABSTRACT

The invention relates to a device for stimulation of the lymphatic system through electric excitation. The device comprises a system positioned on the skin at suitable places known to the expert and sends a train of electric stimuli suitable for stimulating specifically the elements of the lymphatic system. Applications include lymphatic drainage in man and animals, especially the horse and the dog.

This invention relates to the technical sector of electric stimulation of certain tissues.

Such techniques consist of sending electric stimuli to selected tissues, generally repetitive stimuli in the form of stimulating sequences capable of causing local stimulation of a given tissue and yielding a beneficial effect.

The invention applies more specifically to stimulation of the lymphatic system for the purpose of improving, stimulating, or restoring lymphatic drainage, on which overall balance of bodily fluids depends, including that of the circulatory and especially venous system.

General

The purpose of the lymphatic or lymphatico-venous system is to transport excess liquids, toxins, and wastes in the body, filter them, and remove them from the general circulation. This is the body's purification system; the ducts are not engorged when the lymph circulates properly. On the other hand, when for various reasons (overexertion, fatigue, obesity, age (loss of elasticity of tissues), pathology, exposure to pollution, genetic factors, aggravating factors such as tobacco, etc.), lymphatic circulation slows down, the interstitial tissues become filled with toxins and water. Well-known phenomena are then observed, ones such as

Aging of tissues:
bags under the eyes, wrinkles, flaccidity of the skin in general

Pathology:
lymphostasis from
   aplasia or agenesia
   iatrogenic causes
   overflowing of the lymphatic system, in the event of hyperactivity due to a major venous problem.

Cellulitis:
first stage=venolymphatic stage; the lymph is situated in the layer of fat which is hydrophobic (mobile stage);
second stage=infiltrate; the lymph is situated in septa around cells (establishment stage);
third stage=fibrosis tending toward sclerosis; gelatinization of the second stage involving, progressively, sclerosis of the tissue, states of tension presenting an “orange peel” appearance (stage characterized by low mobility, even sclerosis).

It is therefore important to be able to stimulate this system, in a specific and controlled manner, in order to achieve or improve “lymphatic drainage” in particular, that is, to stimulate the lymphatic ducts in a way well known to the expert by means of cycles of stimulation and relaxation. These operations mobilize the nerve cord by call to the lymph and “massage” to reabsorb the cord.

At the level of muscular tissues an overall distinction is made between white or smooth muscles and striated red muscles.

White or organic muscles are known to be muscles of organs, in particular of the small intestine, the stomach, and other known organs.

Veins as well are known to comprise very little white muscle.

It is also known that smooth or white muscles, which are present to a small extent or not present at all inside the veins (median part or tunica media or mediavenosa) have no striae.

Accordingly, the vein possesses no mobility of its own. It is the red muscles outside the vein which permit exertion of mechanical action on the vein (termed the “vis a latere/vein-pump effect”).

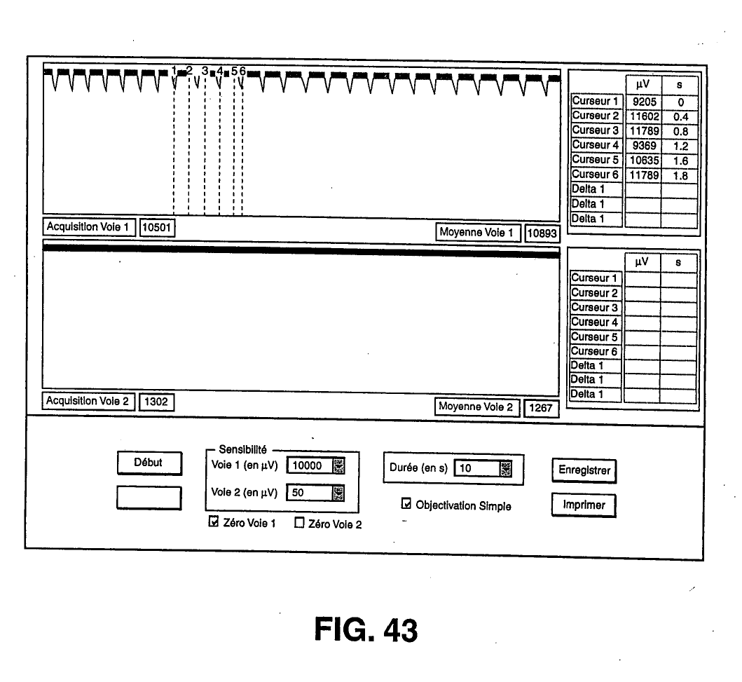

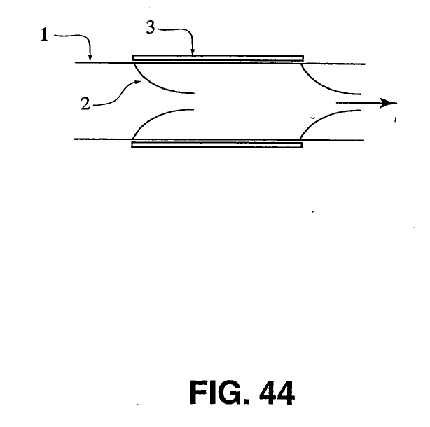

On the other hand, the lymphatic collectors comprise white muscles (see FIG. 44, the “lymphagion,” and FIG. 45). This is one of the major differences from the venous system (no lymphatic varices) and it is this which renders this system dynamizable by mechanical and electric means.

The primary collectors, because of their system of stays connected to the tissue (see FIG. 45), will themselves be dynamized by stretching or depression of the skin;

The lymphatic ganglia are not provided with muscles; hence stimulation of them is much more difficult.

Presented in diagrammatic form in FIG. 45 attached, which consists of FIGS. 45A and 45B, are the “precollectors” at rest (FIG. 45A) and at work (FIG. 45B). It is to be seen that the walls 120 of the precollectors are connected to the conjunctive tissue or “interstitium” (not shown in detail) by “stays” 140.

Because these stays are fixed in the tissues, any depression, any increase in volume, any stretching, will stretch these stays and consequently increase the drainage efficiency of these stays.

Stretching of tissue may be effected by
movement of a member,
increase in volume of a member when edema is present,
adaptation to stress (movement+increase in perfusion and in liquid in circulation).

In the rest stage the walls 120 are not tightly jointed and the “aperture” 130 of the precollector is reduced. In the work stage, in contrast, the walls remain loosely jointed, but stretching of the stays 140 causes increase in the “aperture” 130.

Hence the technical problem of lymphatic system stimulation is radically different from that relating to the venous system.

The waste elimination system, including the lymphatic system, is of the following composition, and its operation is outlined in what follows.

On the basis of an arterial system, which leads into the tissues of food substances, oxygen, and liquids, there is a system for elimination of wastes and renewal of liquids.

The evacuation system is dual in nature:
The venous system: system for rapid regeneration of liquids, minor waste substances (CO₂, etc) with the osmotic pressure and onchotic pressure in tissue maintained in accordance with Starling's Law 1 and 2).
The lymphatic system: much slower system of management of liquids and elimination of major wastes; it also participates in immunology by transporting the lymphocytes which form part of the immunity system.

Lymph is the tissue absorption liquid. 80% of this lymph is situated between the epidermis and the surface muscle aponeurosis.

The lymphatic network represents 500 m² of exchange and evacuation area (system cut flat).

The lymphatic system is the true manager of hydration and also the “scavenger” of these tissues; it also participates in immunology, as explained above.

In its tissues this system is made up of a majority of muscles, in contrast to the venous system.

The lymphatic lattice proceeding from vascular fissures is made up of a terminal dendrite of primary collectors with direct or indirect openings made up of loosely joined cells linked to the tissues by stays; any increase in volume, any shifting of the tissues, stretches these stays, opens these tubules, and the lymph is drawn into the system as a result of increase in pressure.

The following stage enables the lymph to circulate in precollectors, the lymphatic tubule begins to become endothelialized, and ultimately the lymph arrives at the level of the lymphatic collectors.

The lymphatic collector is a separate organized vessel as shown in FIG. 44. This collector is provided with valvulae which send the lymphatic current in a mandatory direction. Between these valvulae a peristaltic wave, a muscular contraction of each lymphangion, passes with a slow automatic rhythm (with the possibility of sympathetic and parasympathetic variations) in the distoproximal direction through the tubular tissue made up of white muscles (subject to the sympathetic and parasympathetic neurovegetative system).

These collectors arrive at the ganglia (afferent vessels).

These ganglia are not muscles; what is involved is a veritable “waste recycling plant,” and the ganglia operate by absorbing the lymph in the ganglionic tissue.

Two lymph cleaning systems are:
phagocyte system=phagocytes “digest” the inert elements (having no genetic material) to produce simple molecules,
immunity system=recognition of a different genetic material (bacteria, viruses, metastases, . . . ); the macrophages and lymphocytes “kill” these different cells and disorganize the DNA in order to sever the amino connections and return to circulation simple non-combined acids, ones which are inoffensive and may even be reused by the body to produce new DNA chains in its own image.

A system of adaptation to stress consists of twofold circulation, slow and fast:
slow=see above
fast=one part of the lymph is treated not on a first ganglion but on the following ones (the one treated by slow circulation at the outset is then shifted to fast circulation). On arrival at the heart the lymph is “clean”; the vessels which distribute ganglia are called afferent vessels.

The lymphatic or lymphaticovenous system has the function of carrying liquids and excess toxins in the body, filtering them, and discharging them into lymphatic and blood circulation. It is the body's cleansing system; when lymph is circulating properly, the ducts are not clogged. On the other hand, when for various reasons, such as overwork, fatigue, obesity, age (loss of elasticity of tissues), pathological conditions, exposure to pollution, genetic factors, aggravating factors such as tobacco, etc., lymphatic circulation slows down, interstitial tissues begin to be filled with toxins. Well known phenomena such as formation of bags under the eyes, facial wrinkles, etc., as well as phlebitis, swelling of members, etc., begin to be observed.

Consequently, this lymphatic system is manager of the quality of tissues and of effective immunological defense of the body, the object of the therapeutic aims of the invention.

The following patents in this field are known:

FR 2 541 119 (Klotz), whose object it is to stimulate the smooth muscles by progressive impulses, or U.S. Pat. No. 4,177,819, or FR 2 433 950, or FR 2 528 709, or U.S. Pat. No. 4,068,669, or U.S. Pat. No. 3,645,267, or U.S. Pat. No. 3,050,695, or U.S. Pat. No. 3,0077,884, or, lastly, U.S. Pat. No. 4,167,189, which measures impedance and applies a sine wave signal, and EP 0 057, EP 0 148 312, EP 0425 673, and FR 2 704 151 whose object it is to measure impedance.

In the opinion of the Applicant, the proximate prior art is represented by the following patents: FR 2 541 119, FR 2 704 151, U.S. Pat. No. 4,167,189, EP 0 425 673. These technologies are presented as stimulating the white (smooth) muscles of the venous media and acting at all times on the venous system.

Also of the state of the art is patent WO 91 07207, which describes application of a pulsed current for the purpose of lymphatic drainage.

It has now been found that the lymphatic or lymphatico-venous system in man or animal, especially the horse, may be stimulated by means of specific electric stimuli which are to be described in what follows.

The invention relates to a new process and a new device for stimulation of the lymphatic system in man or in animals, the horse in particular, characterized in that such process consists in, or permits, application of at least two electrodes to the skin and causes sequences of electric stimuli to pass into the human or animal body involved (hereinafter referred to jointly and severally as “the body”), each stimulus or pulse being characterized in that
the period of excitation is shorter than the period of rest or relaxation;
the period of excitation ranges from 2 to 8 milliseconds;
the period of rest or relaxation ranges from 400 to 850 milliseconds.

In one particular application, the intensity of the electric current applied is lower than or equal to 1 mA (milliampere), and preferably of the order of 6 to 300 microamperes (mA) or more, depending on the number of electrodes which, along with the patient's “feeling” (physical response), represent the only restrictions imposed in this context.

In a preferred embodiment pulse trains or “bursts” of 5/5 to 10/10 or 15/15, preferably 10/10, are to be applied.

Each train or sequence of pulses exhibits reversal of polarity relative to the preceding sequence. Consequently, the mean value of the current is zero; this prevents any polarization of material in the tissue treated.

In a specific embodiment, the frequency of each pulse is to range from 0.1 to 3 Hz, and preferably from 0.7 to 2.5 Hz, preferably in the vicinity of 1.5 or 2 Hz.

Other characteristics and advantages of the invention will be better understood by reading of the following description and by referring to the tests and to the attached drawing, in which

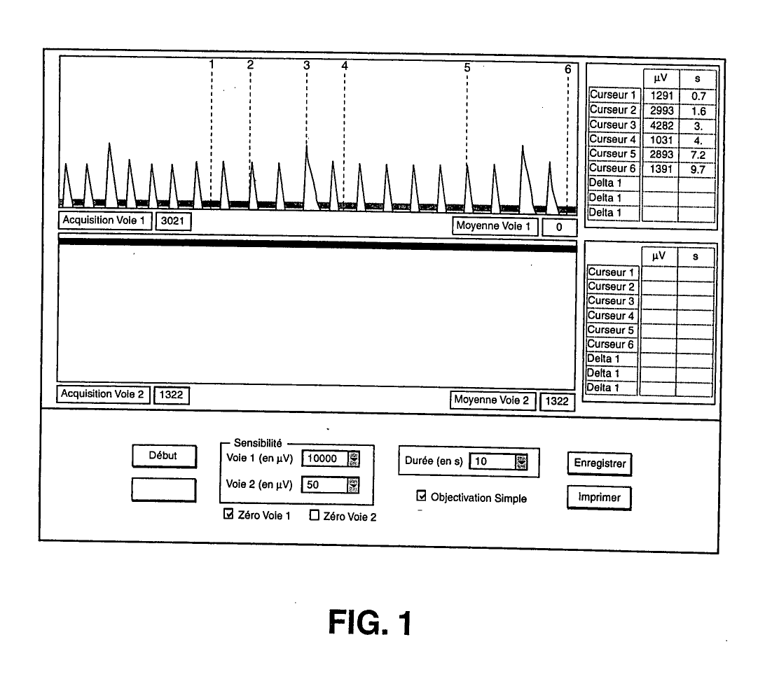

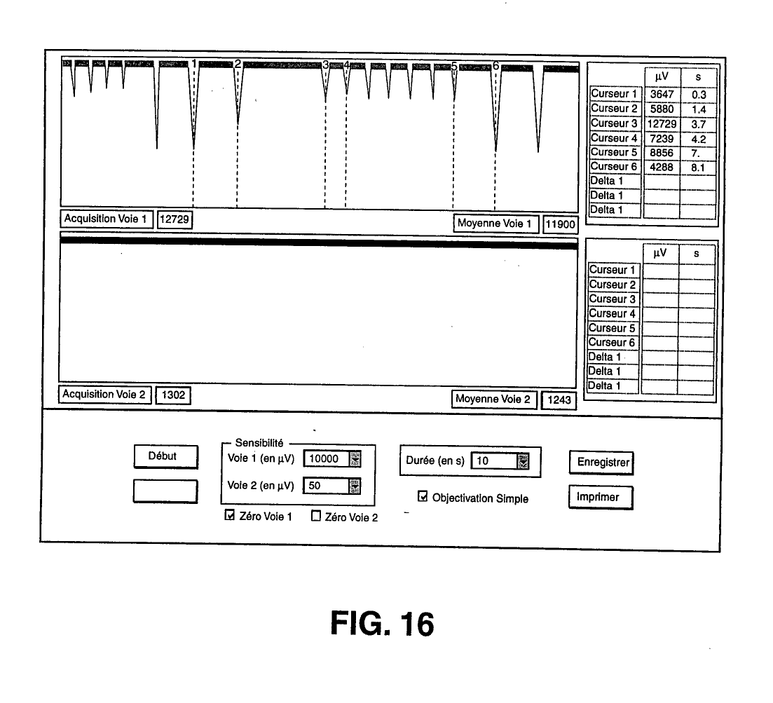

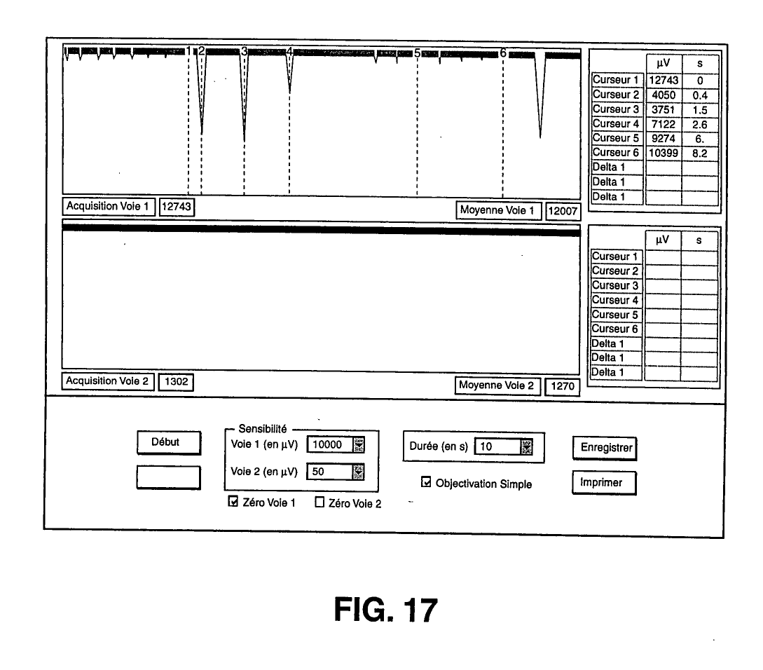

FIGS. 1 to 43 present the curves of EMG activity obtained with the device claimed for the invention and with devices of the prior art, as specified in the TESTS presented below.

FIG. 44 represents a “lymphangion.”

FIG. 45, which consists of FIGS. 45A and 45B, presents the structure of the tissues around the lymphatic system.

### TESTS

General Mode of Operation

Three anatomical parts were taken from swine recently sacrificed (5 minutes earlier): the heart, certain blood vessels, the lungs, and the small intestine.

Dissection was performed and the following anatomical elements were selected:

| | |
|---|---|
| vena cava | 10 cm/40 g |

thoracic canal (canalis lymphaticus major) and small intestine making up a total of 150 g

| | |
|---|---|
| red skeletal muscles | 150 g |

Tests were also conducted with healthy humans (referred to as “SUBJECT” in what follows).

The tests were conducted with a device of the EMG type with biofeedback (visual graphic inspection of the contraction) of the YSY EST™ type made by the YSY MEDICAL™ company capable of performing EMG measurements (activity—electromyogram) and comprising a system of filters designed to register only the activity of the tissues (especially contractions) and not interfering signals.

The pulses were applied, as appropriate, either with needles planted in the test tissue (referred to as “ACU” in what follows) or by alligator clips clamping the test tissues (“CROCO”).

This testing procedure has been deemed to be suitable for proper simulation of “external non-invasive” stimulation procedures by contact electrodes, which are the ones employed in practice, except in precisely identified particular cases.

The purpose of the tests was to compare the effects of different devices at different settings in order to determine the forms and characteristics of current suitable for treating or stimulating the lymphatic system in accordance with the objectives cited in the foregoing.

Activity was calculated by means of a formula determining the mean of positive and negative pulses established by taking account of, and relative to, the “path acquisition” which is displayed in the window to the left of the curves, which will be understood by the expert.

TEST No. 1

Comparative test of Datavein™ and the FDLP device claimed for the invention.

The device is set in accordance with the operating instructions for the Datavein™, that is, at 1.75 Hz (frequency of each pulse), work time of 4 millisec, intensity of 6 μA, and a rest period of 567 millisec, with 8/8 electric pulses.

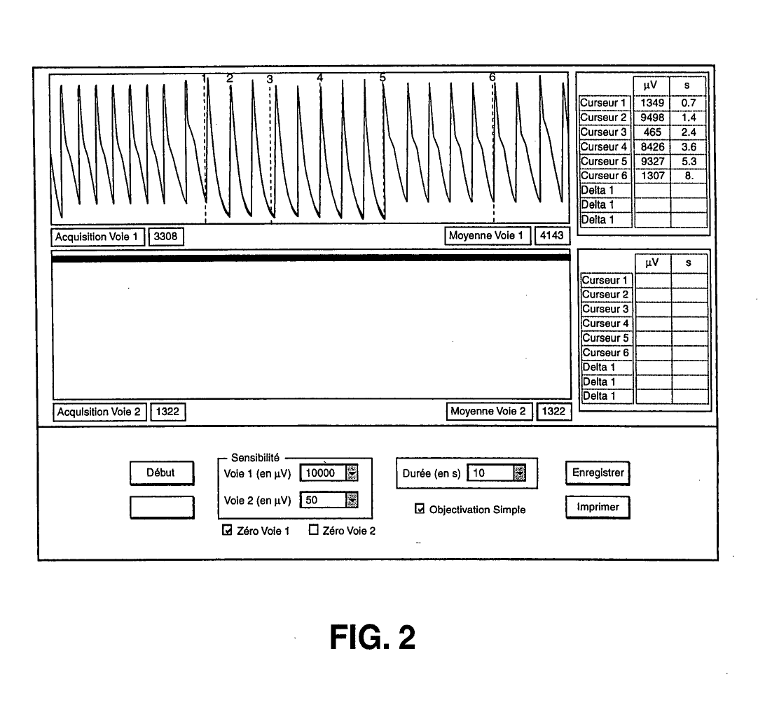

FIG. 1 presents the EMG results obtained with the Datavein™ device, and FIG. 2 the results obtained with the FDLP device at the same settings.

The positioning of cursors permits calculation of representative and reproducible averages.

It is to be seen that average EMG activity is 2900 for the Datavein™ and 4143 for the FDLP, that is, activity approximately twice as high for the second device, at the same settings.

The widely differing shape of the curves is also to be noted.

TEST No. 2

The Datavein™ and FDLP devices were compared.

The FDLP was set to the values recommended for the Datavein™ (identified by the reference “FDLP DATA”).

The test was conducted with lymphatic tissue or of the lymphatic type of swine (lymphatic tissue and small intestine (referred to as “LYMP”) and by means of alligator clips (referred to as “CROCO”).

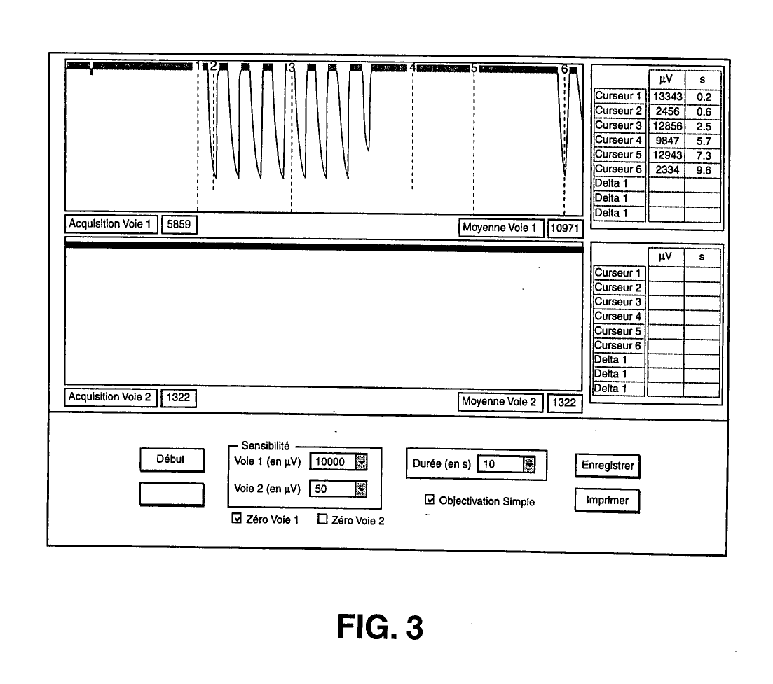

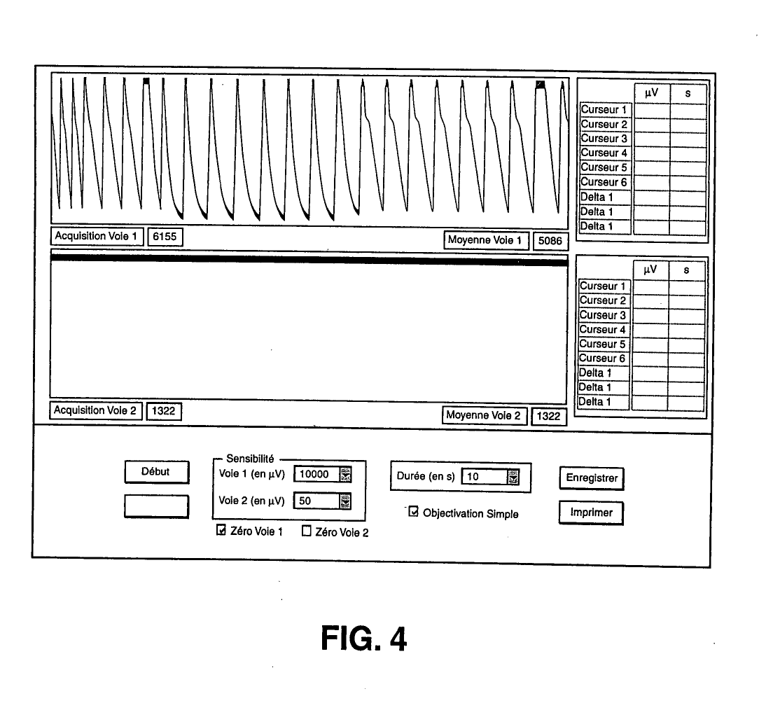

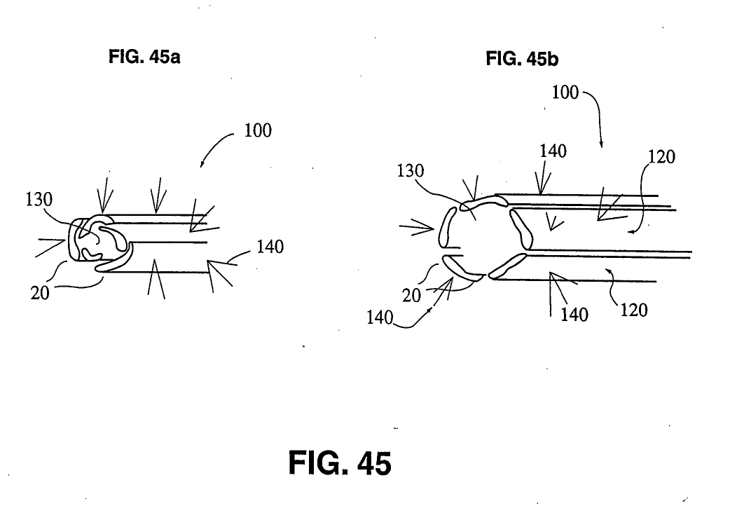

The EMG activity measured with the Datavein™ is 1014 (FIG. 4b) and 10971 (FIG. 3) with the FDLP, which is seen to yield an effect ten times greater.

Note is also to be made of the different form of the pulses, and, with the Datavein™, regular pulses are observed, while pulses alternating in groups of eight with inversion of polarity are observed with the FDLP, but not with the Datavein™.

TEST No. 3

The Datavein™ and FDLP devices were compared.

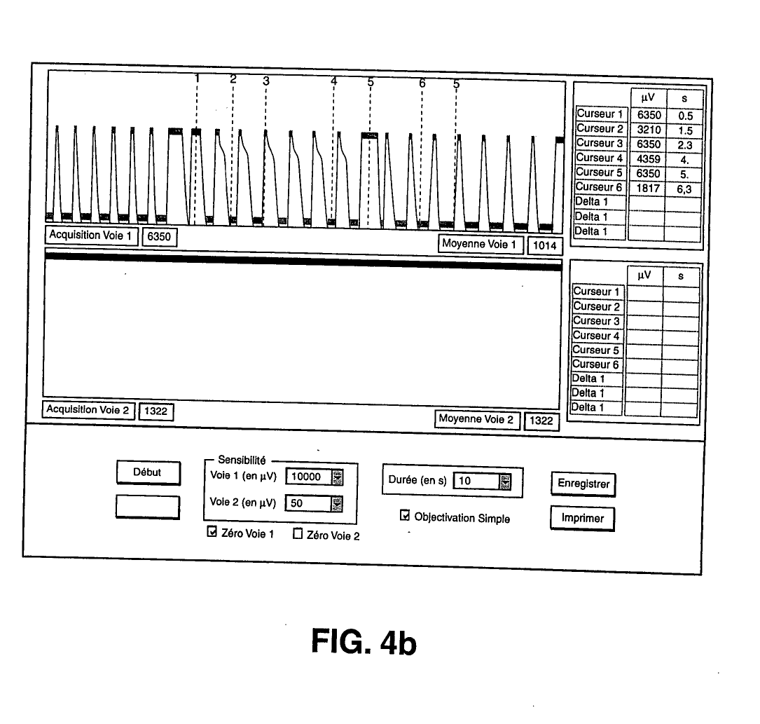

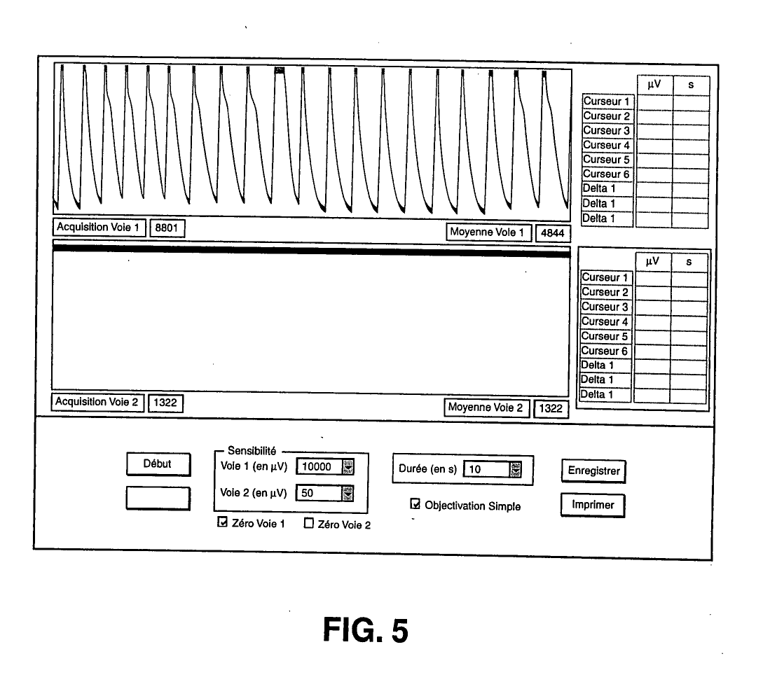

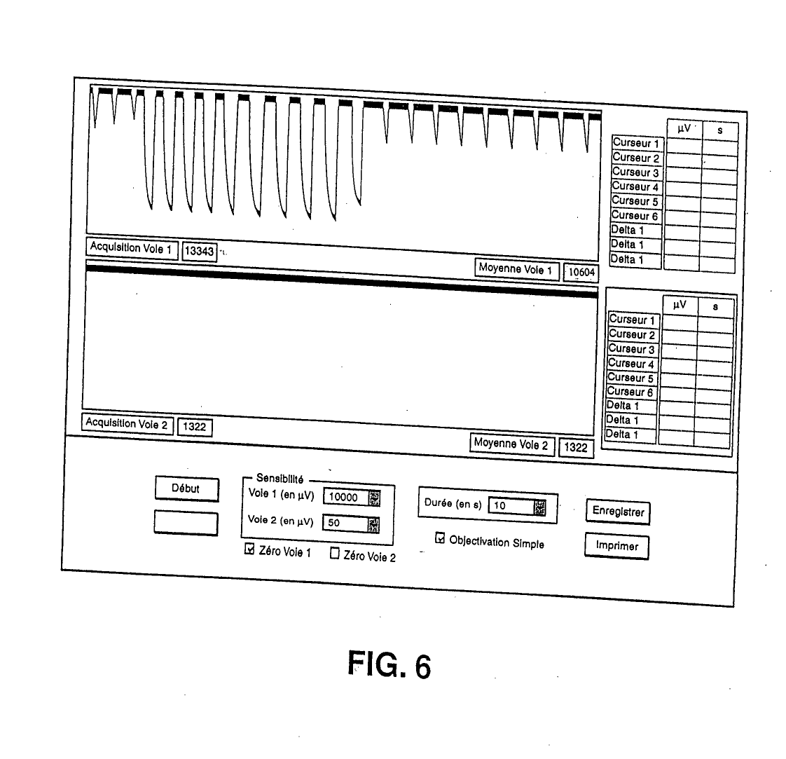

The test was conducted with a human being (“SUBJECT”) by means of the Datavein™ with two tests (FIGS. 4 and 5) and the FDLP with the Datavein™ settings (FIG. 6) and thus more than with the Datavein™ (FIG. 7).

The devices were set to 30 μA.

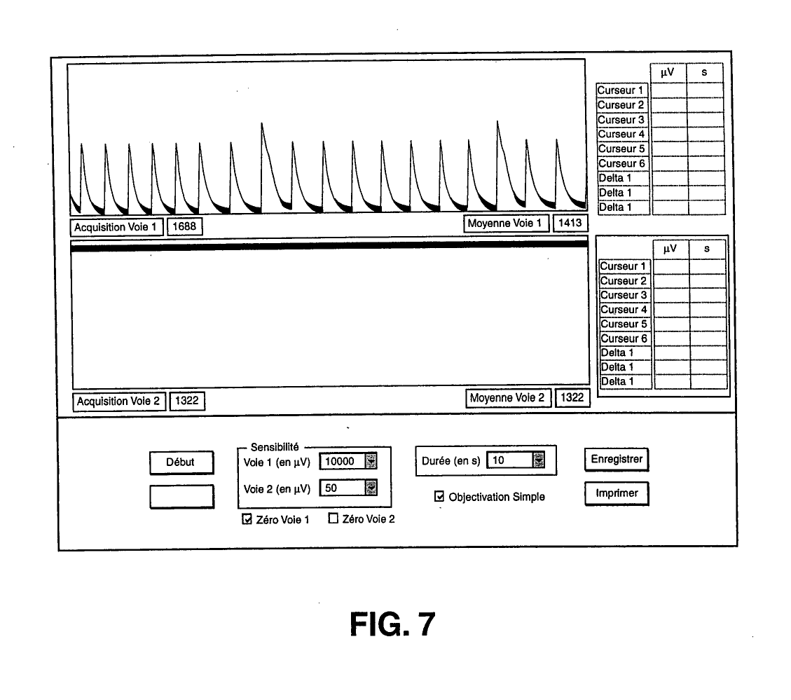

It is to be seen that the activity measured with the FDLP with the Datavein™ setting is 10604 (FIG. 6), while that measured with the Datavein™ is 1413 (FIG. 7), 1554 (FIG. 8) 5086 (FIG. 4), 4844 (FIG. 5).

Hence the FDLP is 2 to 7 times more powerful than the Datavein™ in this test.

TEST No. 4

An effort was made in this test to compare the physical “feeling” of a human subject when treated with the Datavein™ or with the FDLP.

It was found that, in order to find the feeling of the FDLP set to only 30 μA, it was necessary to increase the intensity delivered by the Datavein™ to 150 μA.

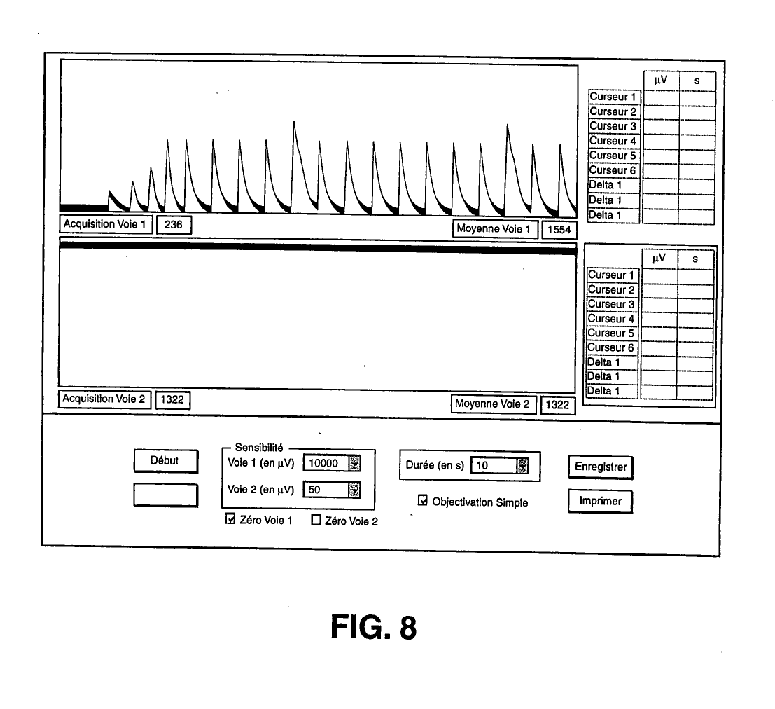

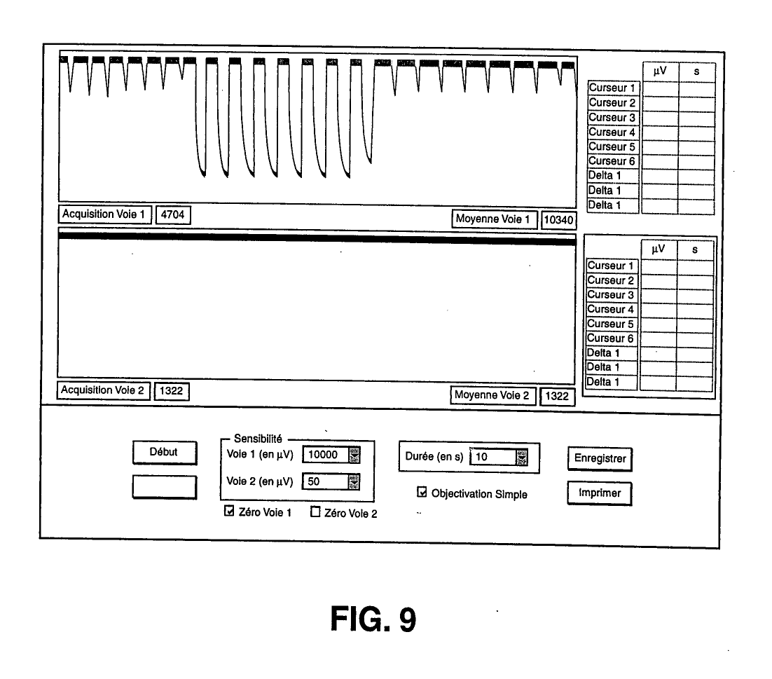

This is confirmed by FIGS. 9 and 10 (Datavein™) set to 154 μA; the average EMG is 10340 or 10616.

It is useful to make a comparison with FIG. 6 (FDLP set to the parameters recommended for the Datavein™), which delivered a mean activity of 10604 at 30 μA, while the Datavein™ produced only 1413 at the same intensity.

TEST No. 5

The overall superiority of the FDLP having been demonstrated above in various configurations, this test was undertaken to investigate the best current characteristics for the FDLP.

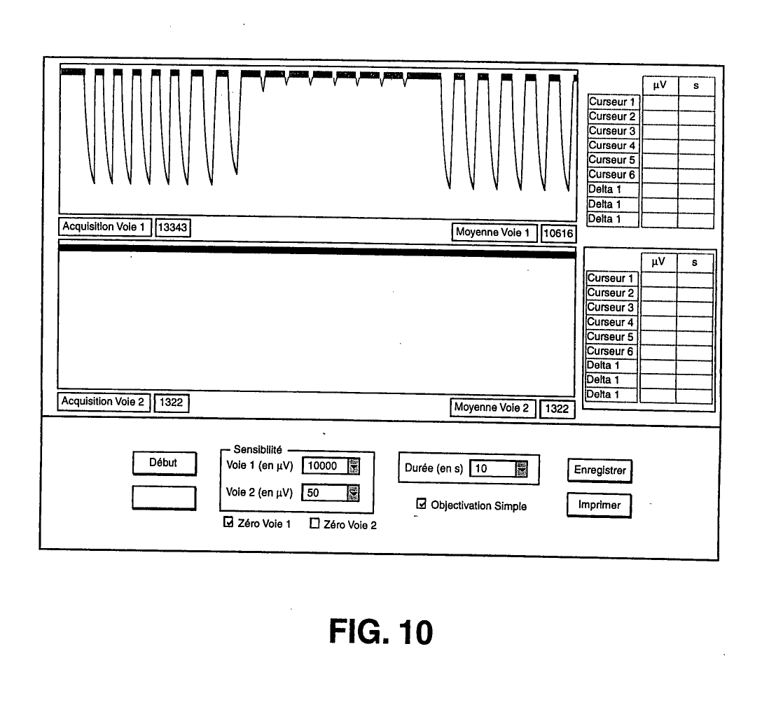

FIG. 11 illustrates the activity curve in a subject of the FDLP set to 6 millisec work time, 500 millisec rest time, 10/10 sequences or “bursts,” and 6 μA intensity.

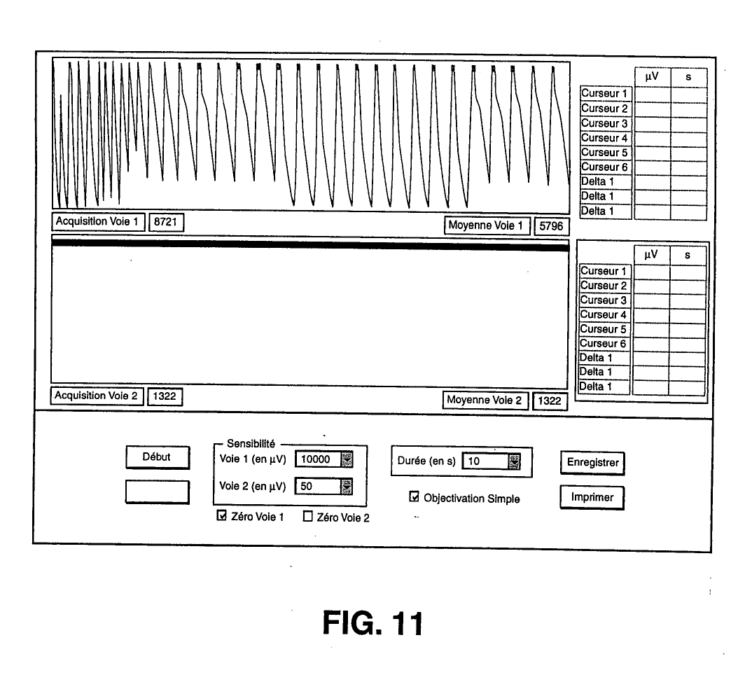

FIG. 12 shows the same curve, except that the sequences are 5/5 rather than 10/10.

A mean activity of 5796 is observed for the 10/10 curve, in contrast to 5343 for the 5/5 curve.

The two activities are thus more or less equivalent, but the setting at 10/10 produces a more comfortable “feeling” than at 5/5.

An attempt has been made to explain this phenomenon and, without wanting to be tied to any theory, the applicant believes that the 10/10 setting is better suited to the slow rhythm of the natural oscillations of the lymphatic system. In order to stimulate a ganglion manually, the expert will perform 6 to 8 oscillations per minute, which corresponds to a frequency of the order of 0.1 Hz. A lymphatic collector performs 10 12 movements per minute, at a frequency of the order of 0.2 Hz.

The FDLP 10/10 setting, with 6 millisec of work and 650 millisec of rest, at 6 μA, yields a frequency of the order of 1.52 Hz.

The FDLP 10/10 setting, with 2 millisec of work and 500 millisec of rest, at 6 μA, yields a frequency of the order of 1.99 Hz.

It appears that the combination of these two frequencies and pulse trains harmonize with the natural slow rhythm of the lymphatic system.

The setting recommended for the Datavein™ is 1.75 Hz.

At the same setting the Datavein™ yields only values of 2900 or 1413 (FIG. 1 at 6 μA and 7 at 30 μA), and accordingly in this test reveals an efficiency even less than one-half that of the FDLP.

TEST No. 6

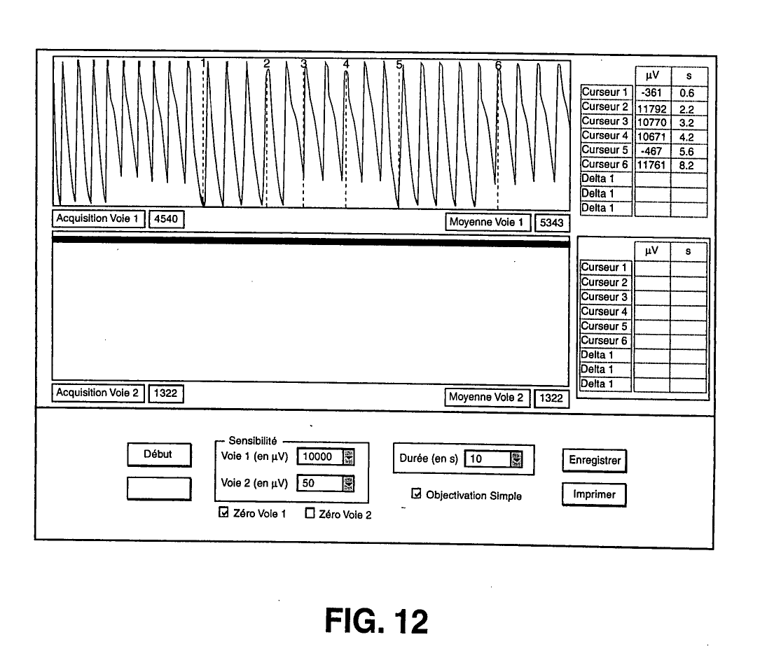

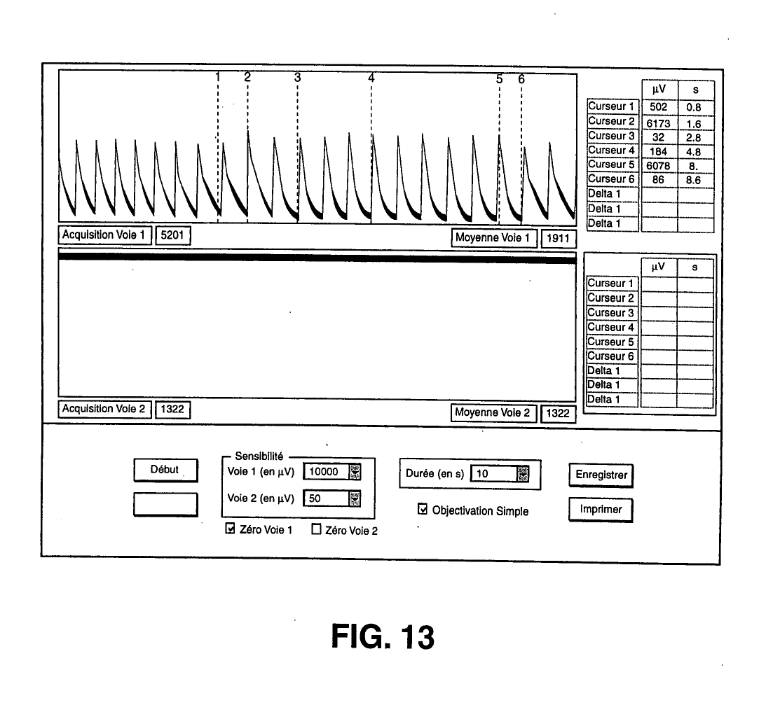

Comparison was made again in this test of two different FDLP settings, one (FIG. 13) with a human subjects, with 2 millisec of work, 650 millisec of rest and 10/10 sequences, at 6 μA, and the other (FIG. 14) with a human subject with 2 millisec of work, 500 millisec of rest and 5/5 sequences.

The mean efficiency obtained in FIG. 14 is 677, as against 1911 in FIG. 13.

Hence, it appears to be more efficient to provide a long rest time for the same work time.

It also appears to be more interesting to provide sequences of 10/10, this confirming a previous test.

TEST No. 7

The FDLP was tested on a human subject with a setting of 6 millisec of work time, 650 millisec of rest time, and sequences of 10/10, at 30 μA/

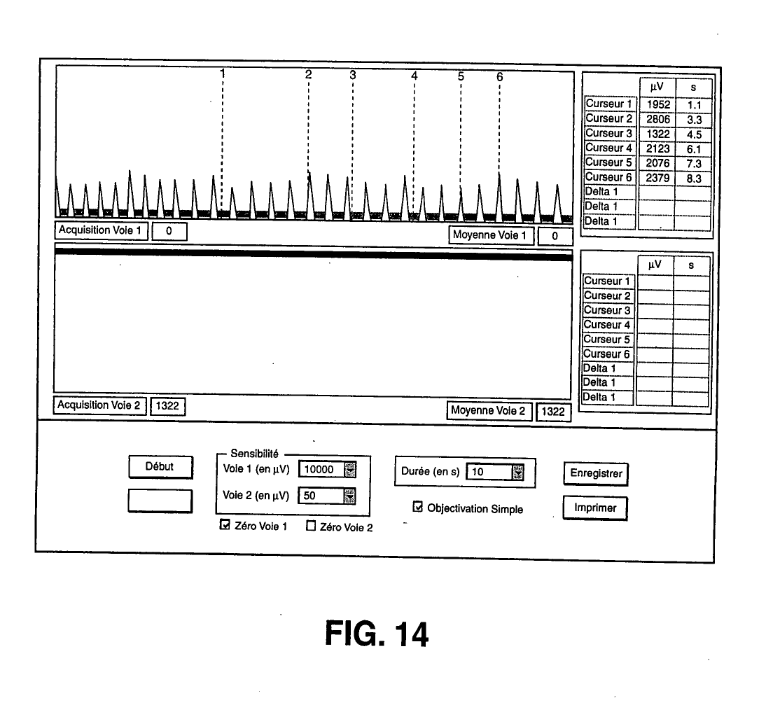

An EMG activity of 8435 was obtained (FIG. 15).

### Interim Finding

The foregoing tests on the one hand reveal efficiency 2 to 10 times greater for the FDLP than for the Datavein™, and, on the other hand, indicate the possibility of setting the FDLP device for generation of current of two types:

a first type of current is a “regulator” or “reeducator” current, useful primarily for treatment of pathological conditions of the lymphatic system, one which is centered on a setting of approximately:

| | | | |
|---|---|---|---|
| 2 millisec of work | 500 millisec of rest | 10/10 sequences | >>1.99 Hz |

a second type of “stimulator” or “turbo” current, causing stimulation of the lymphatic system much stronger than the preceding one, useful primarily in other applications of the FDLP, physiological work, and centered around a setting of approximately

| | | | |
|---|---|---|---|
| 6 millisec work | 650 millisec rest | 10/10 sequences | >>1.52 Hz. |

TEST No. 8

Tests were conducted with venous tissue, either with implanted needles or with alligator clips.

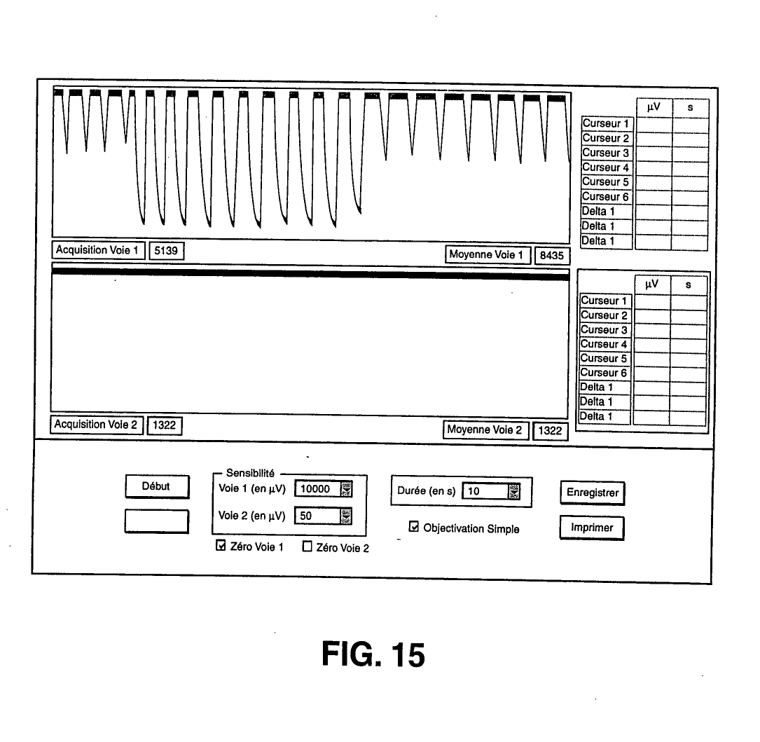

In order to obtain the curves shown in FIGS. 16, 17, and 18 use was made of a FDLP with Datavein™ settings (“FDLP DATA”) with alligator clips (“CROCO) and with venous tissue (“VEINE”). Activities of 11900, 12007, and 12122 were obtained.

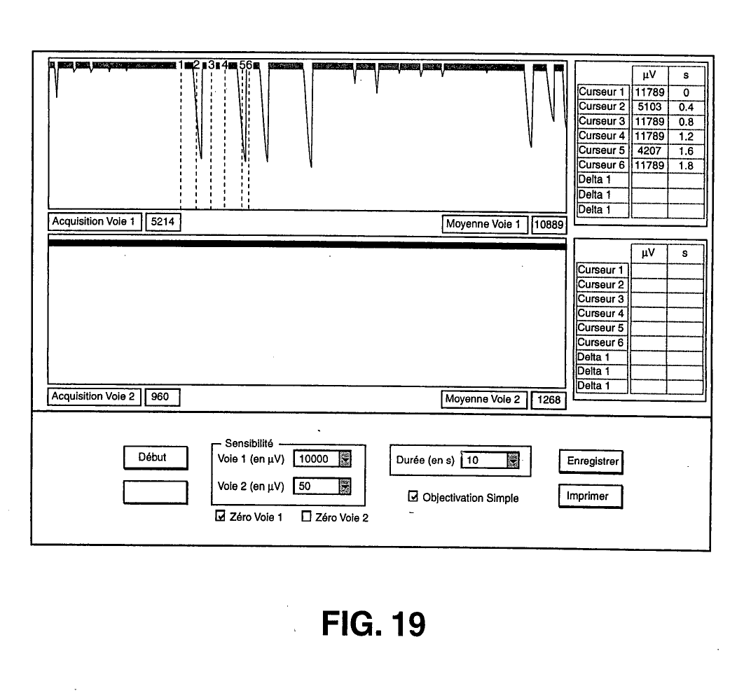

In order to make a comparison with the Datavein™ a test was also conducted with the Datavein™ at the settings recommended by the manufacturer, on venous tissue and with alligator clips (FIG. 20). A value of only 9187 was obtained.

Consequently, the FDLP clearly performs better than the Datavein™ even with venous tissue.

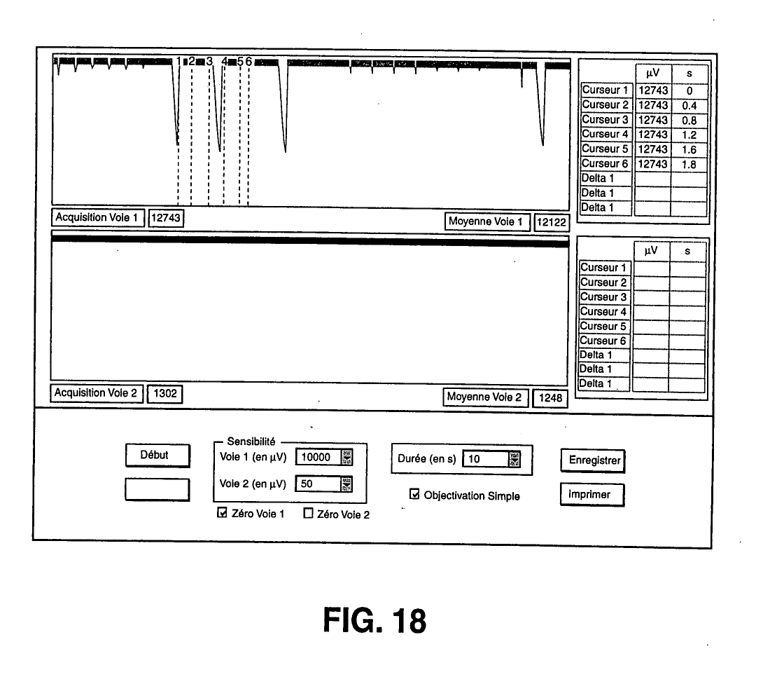

The FDLP with Datavein™ settings was also tested on venous tissue but with needles (“ACU”) implanted (FIG. 19). A value of 10889 was obtained, that is, a value lower than those obtained with the alligator clips. This appears to invalidate the theory of the Klotz patent to the effect that the veins are made up of white muscles. In fact, in a case such as this the ACU effect should have been greater than the CROCO effect.

TEST No. 9

Tests were conducted:

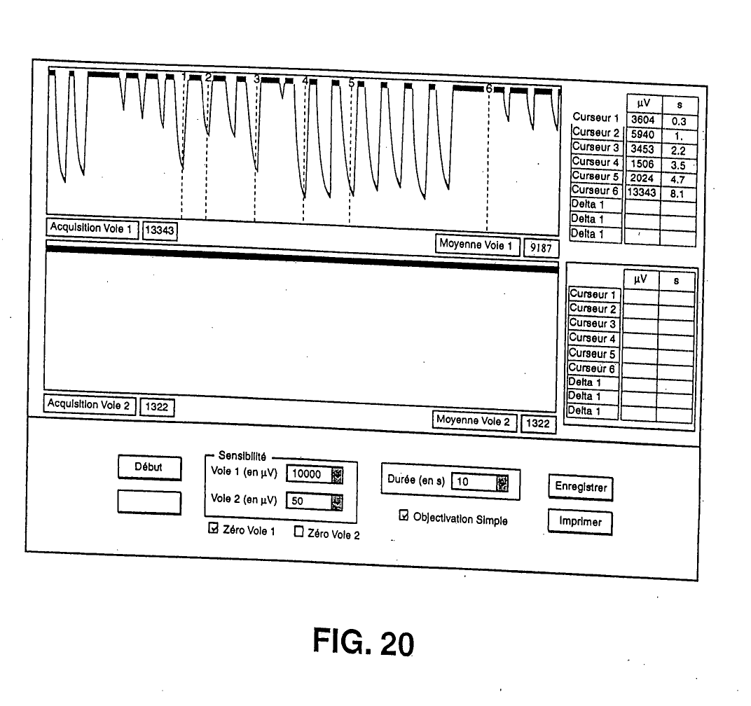

with red muscle using FDLP with Datavein™ settings, with needles (FIG. 21). Activity (mean): 5344.

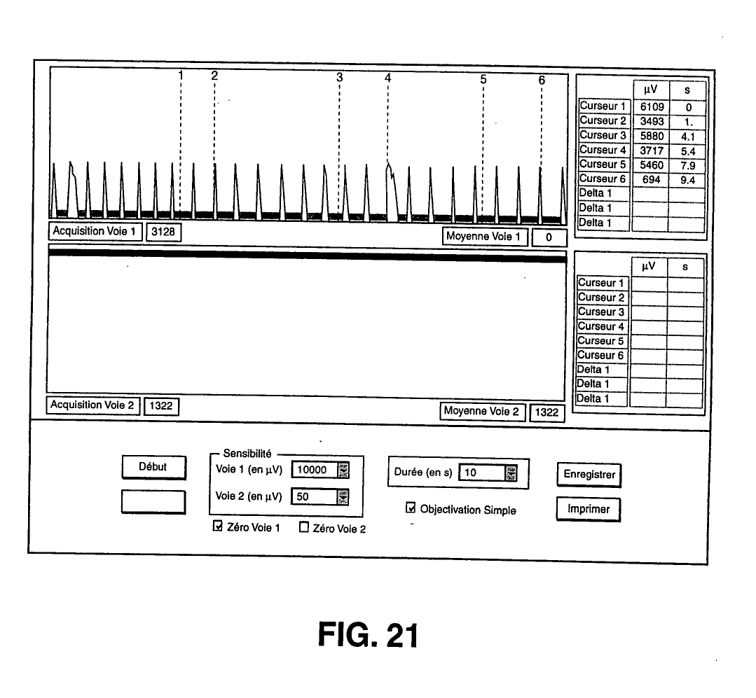

with red muscle using FDLP with Datavein™ settings, with alligator clips (FIG. 22). Activity: 1856.

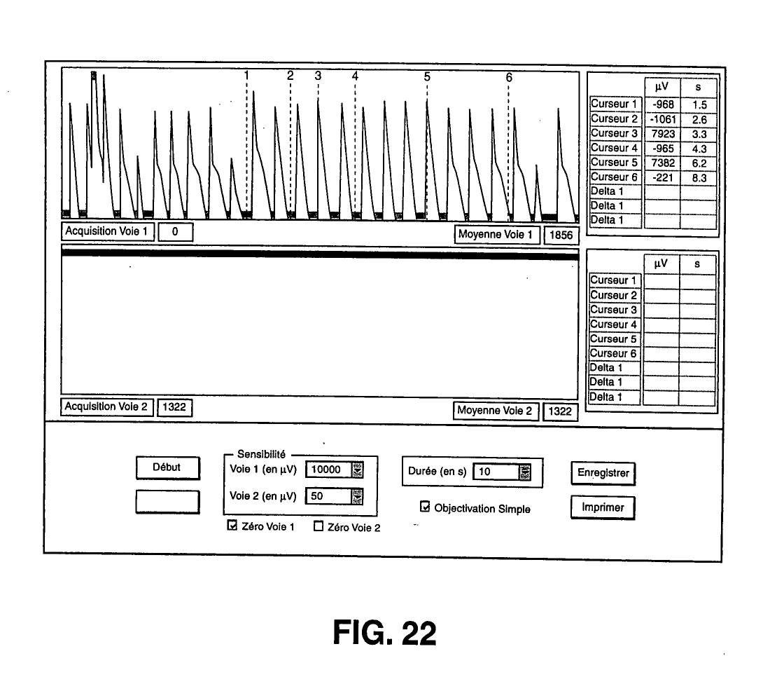

with red muscle using Datavein”, with alligator clips (FIG. 23). Activity: 1172. Hence the result obtained with Dataveimm is not as good as that obtained with FDLP.

TEST No. 10

“Regulator” current tests were conducted with the FDLP:

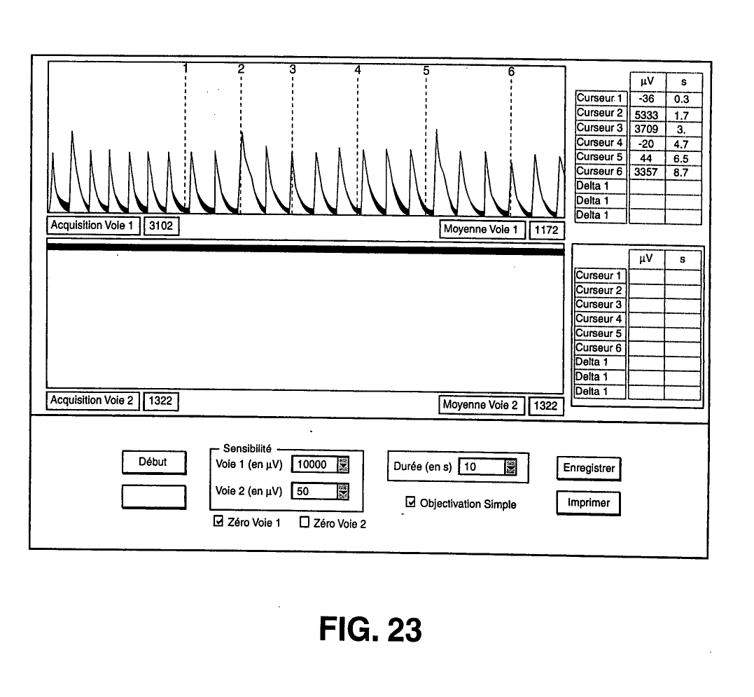

with red muscle, using the FDLP set for 2 millisec work, 500 millisec rest, needles, and 10/10 trains (FIG. 24): activity: 2730.

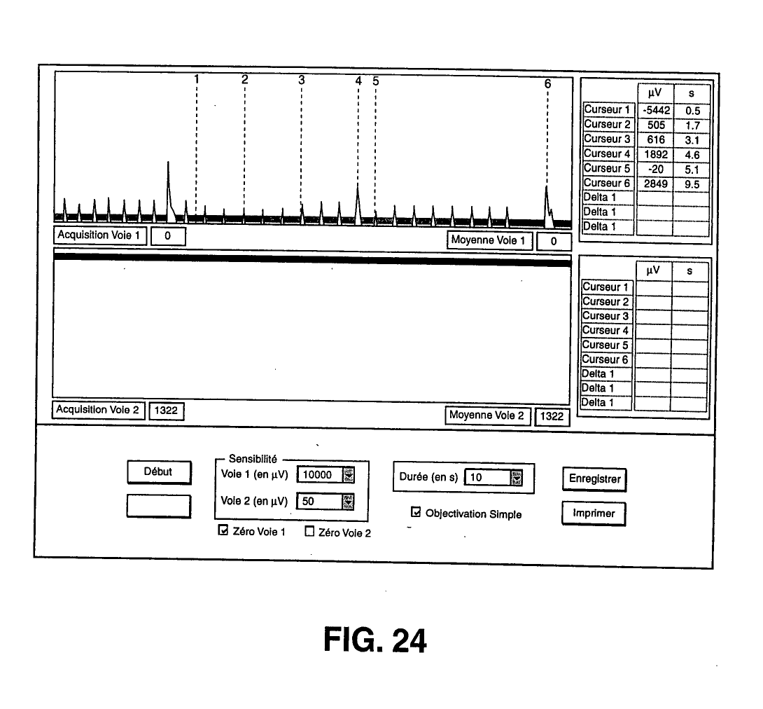

with red muscle, using the FDLP set for 2 millisec work, 500 millisec rest, needles, and 5/5 sequences (FIG. 25): activity: 3215.

It is clearly to be seen from the curves that the 5/5 setting is superior to the 10/10 setting for the red muscle, while the opposite finding applies to the lymphatic tissue (performance with 10/10 setting higher with lymphatic tissue).

TEST No. 11

Square wave signal tests were conducted.

Use was made of the MICROSTIM™ device of the Physio-INSEP™ company, the manufacturer of which asserts that the device operates as a “vein pump” or by the effect of pumping on the striated red skeletal system peripheral to the venous system (by contraction-relaxation), at an initial setting of 1.25 Hz.

The following curves were obtained:

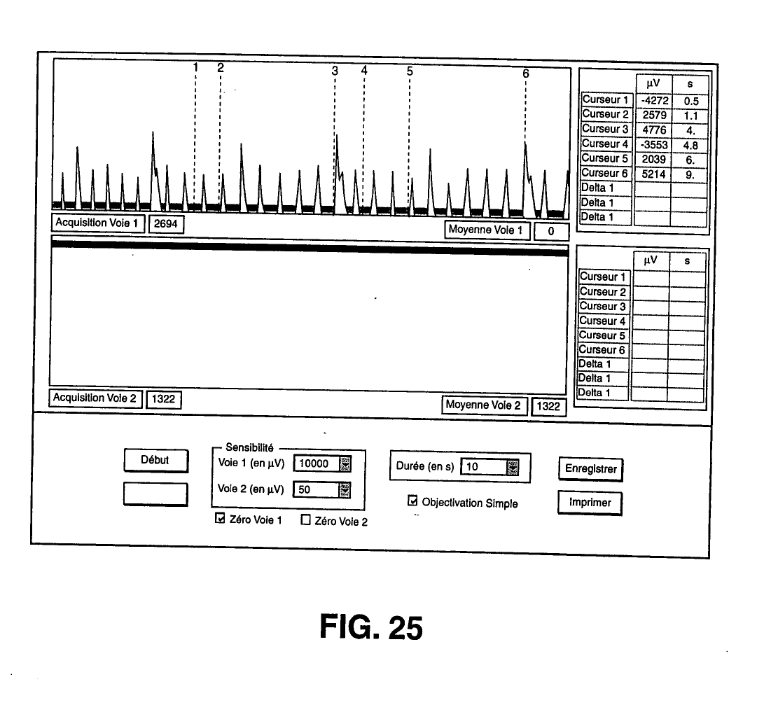

Croco with lymphatic tissue (FIG. 26); result mediocre

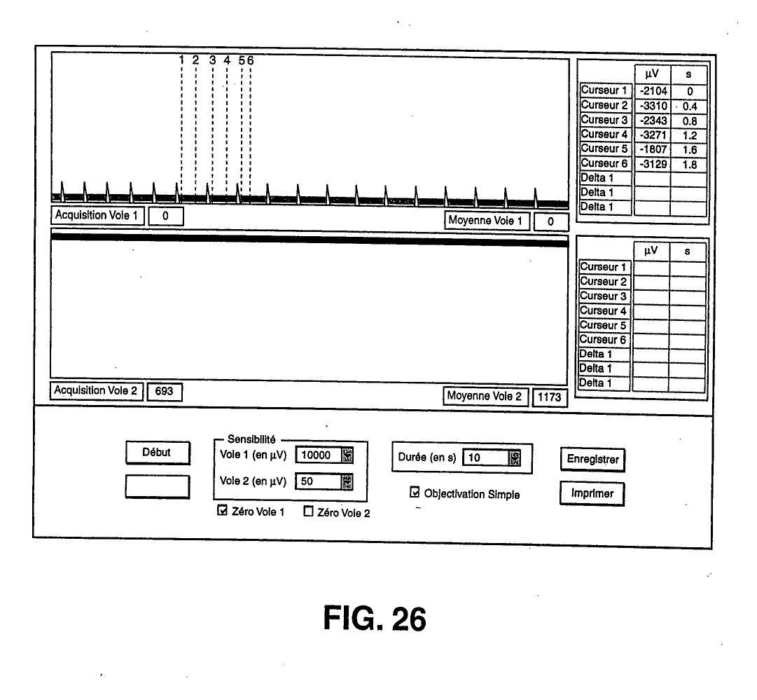

Needles with lymphatic tissue (FIG. 27); result somewhat better

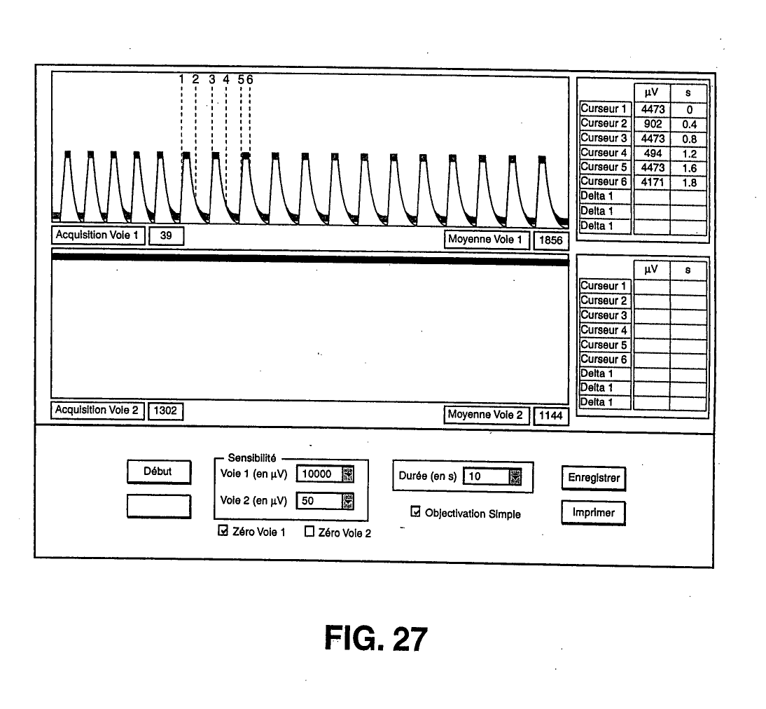

Croco with red muscle (FIG. 28); good result (3554)

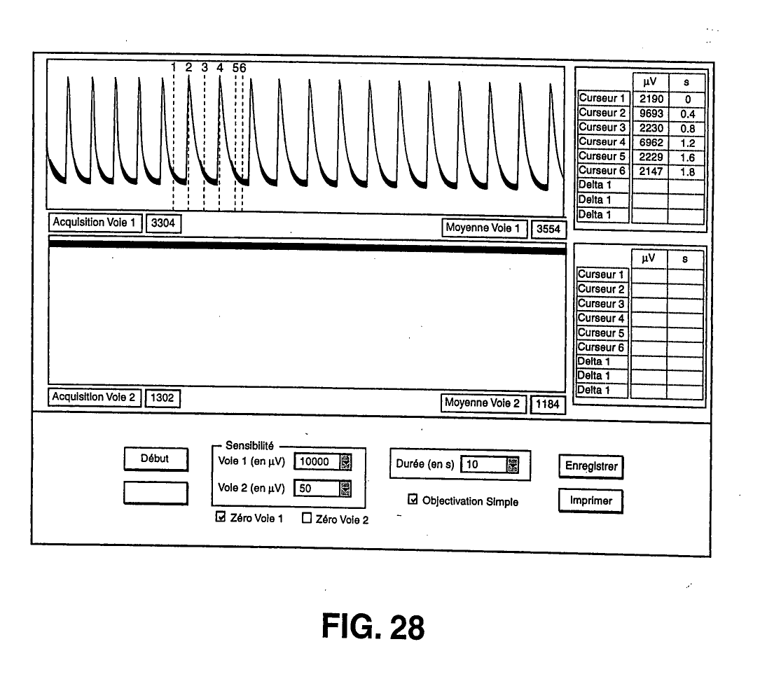

Needles with red muscle (FIG. 29); good result (2323)

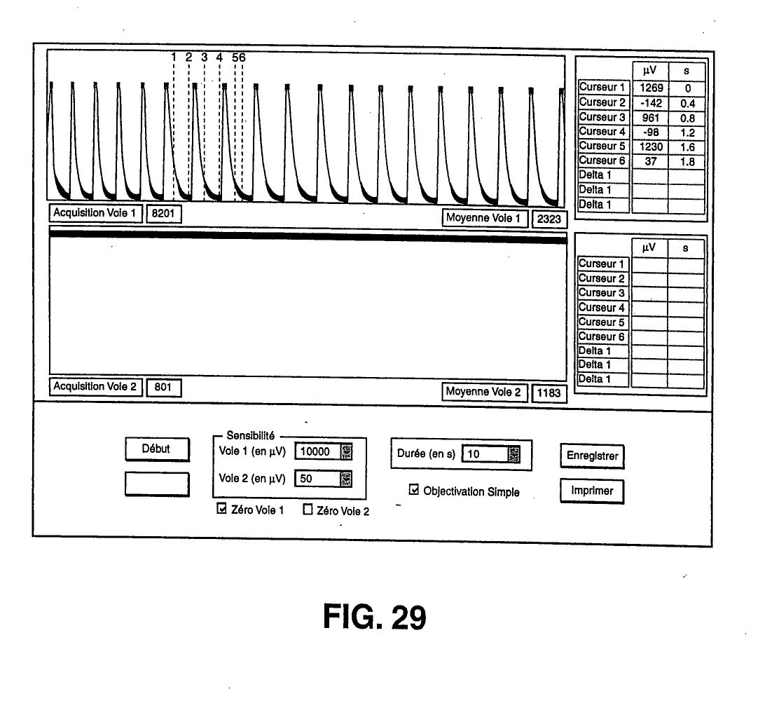

Croco with venous tissue (FIG. 30); 9567

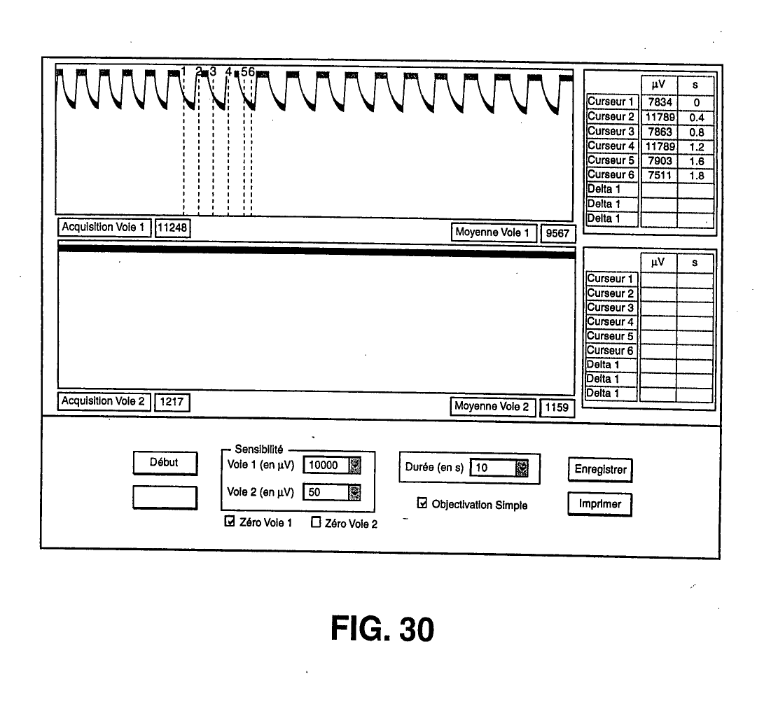

Needles with venous tissue (FIG. 31); 1584

The good results reflected in FIGS. 28 to 31 were predictable, because the device is designed for “vein pump” operation by stimulating the perivascular skeletal striated muscles.

The MICROSTIM™ device was then used at a second setting of 1.50 Hz.

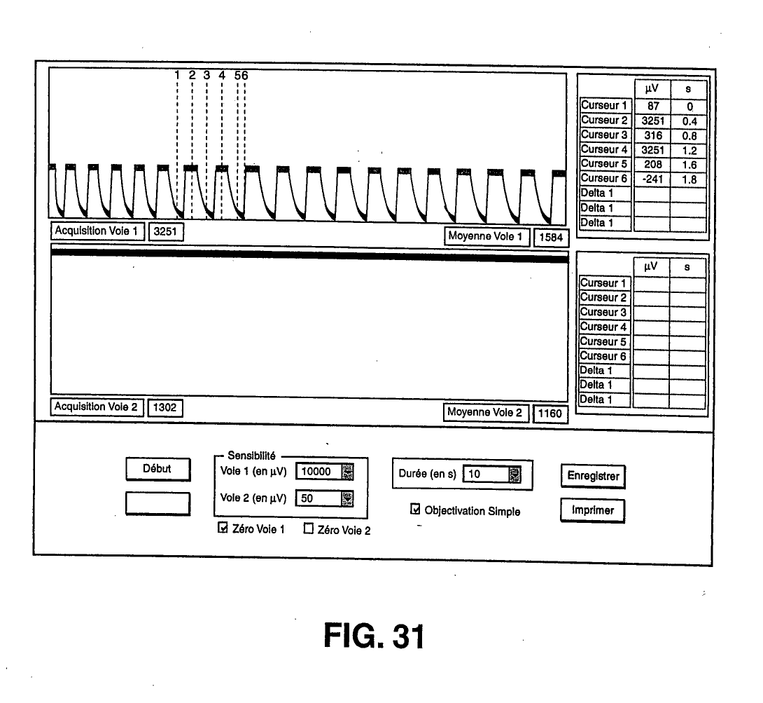

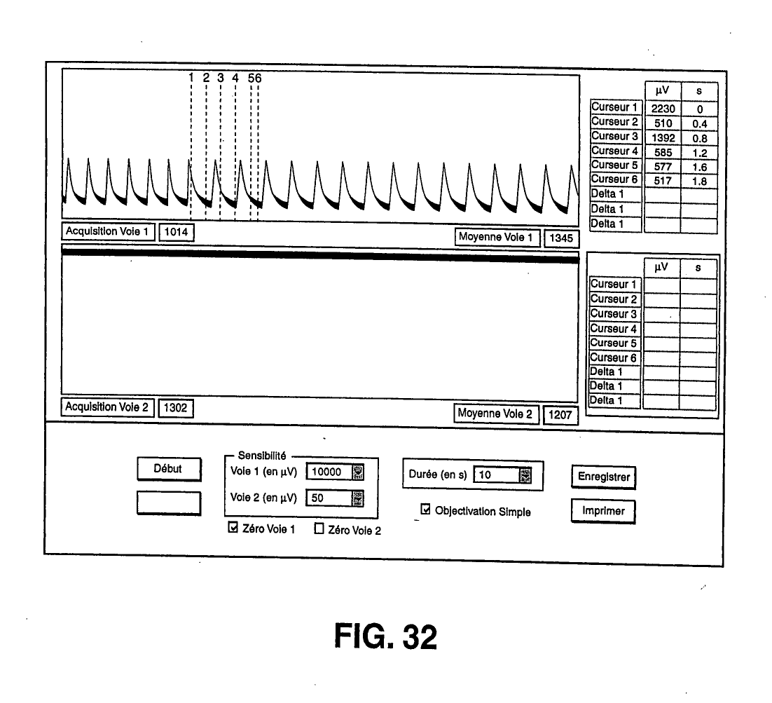

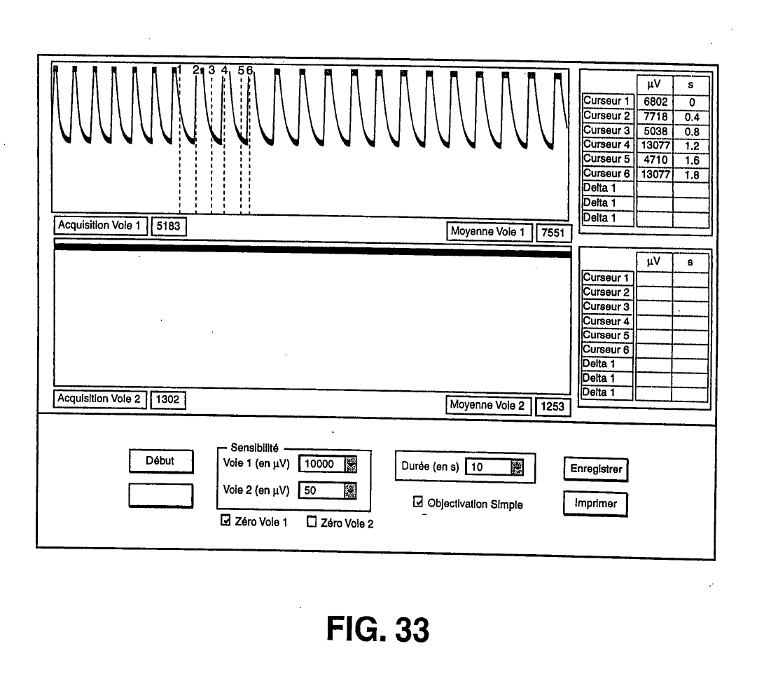

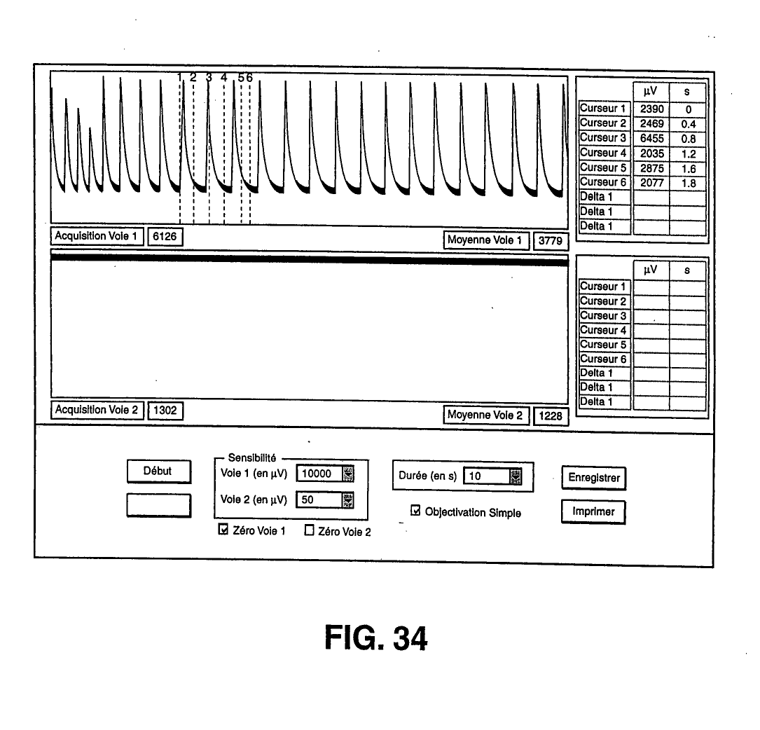

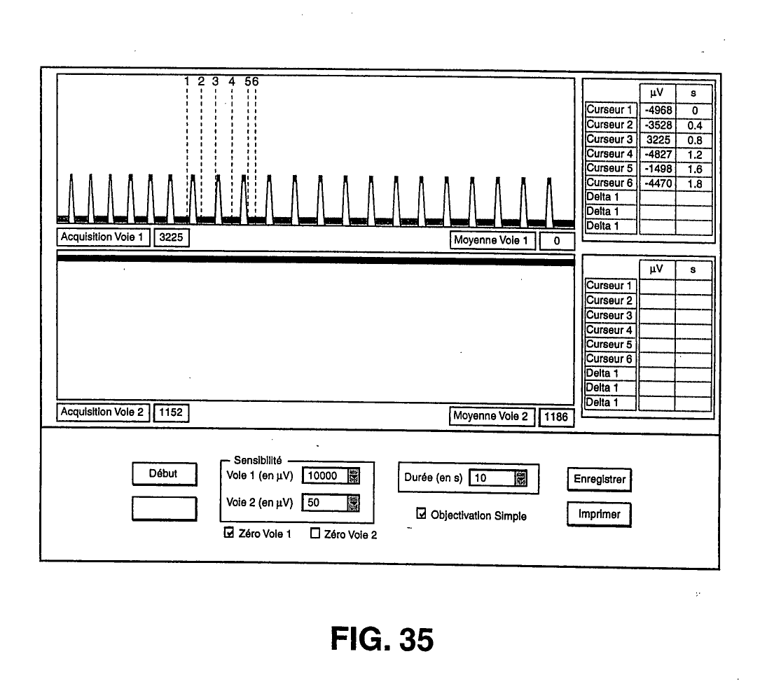

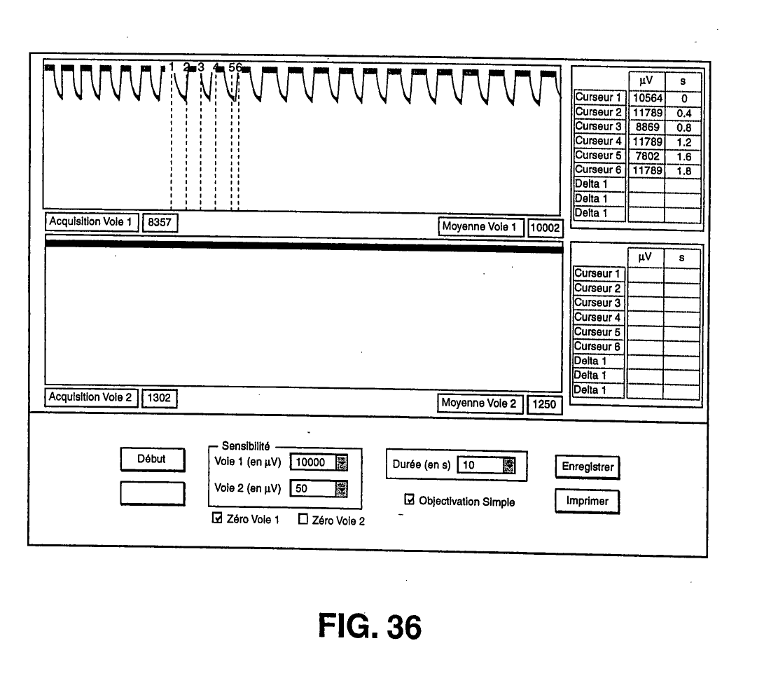

The following curves were obtained:

| | |
|---|---|
| Croco on lymphatic tissue (FIG. 32) | mediocre results 1345 |
| Needles in lymphatic tissue (FIG. 33) | 7551 needle problem |
| Croco on red muscle (FIG. 34) | good result 3779 |
| Needles in red muscle (FIG. 35) | good but inferior result |
| Croco on venous tissue (FIG. 36) | 10002 very good result |
| Needles in venous tissue (FIG. 37) | 10901 very good result |

It was inferred that application of square wave signals is better suited for treatment of the venous system, because the stimulation affects the striated perivascular muscles but not at all, or very slightly, the mediavenous, which is almost absent from the venous systems as a whole.

The MICROSTIM™ device was then used at a third setting of 1.75 Hz, which is the setting recommended for the Datavein™ by its manufacturer.

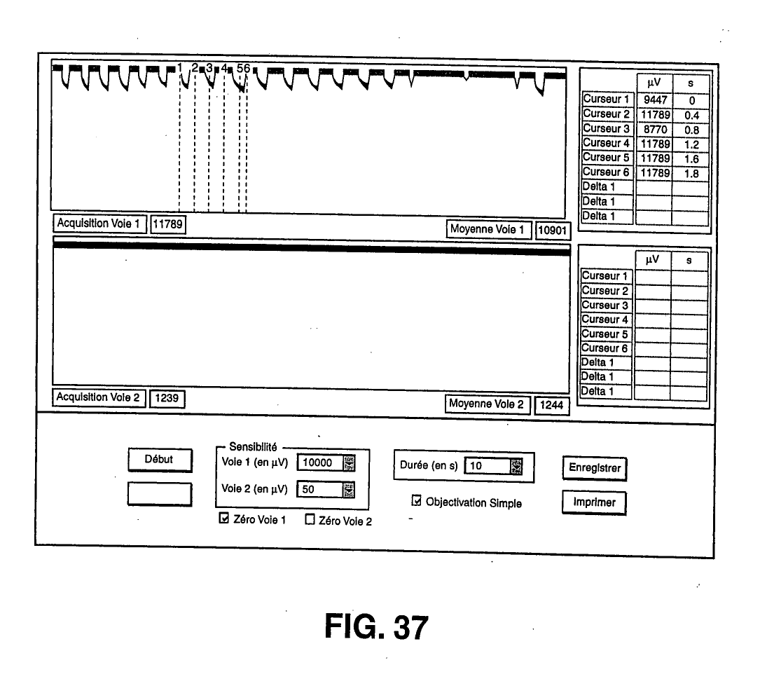

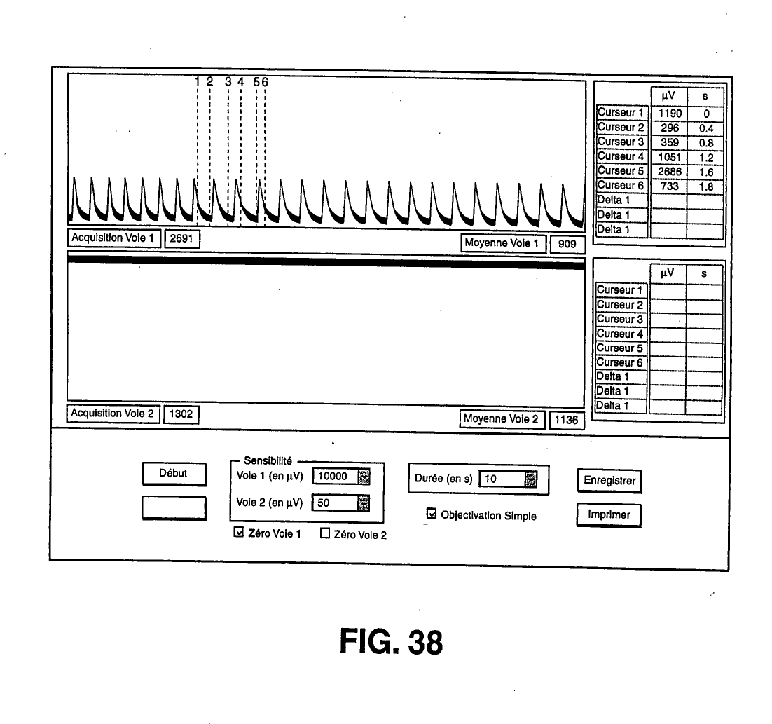

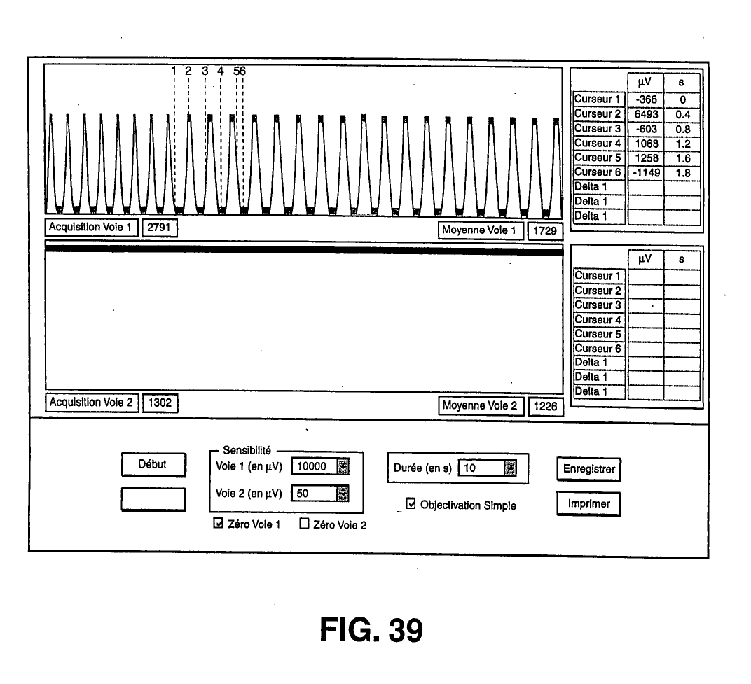

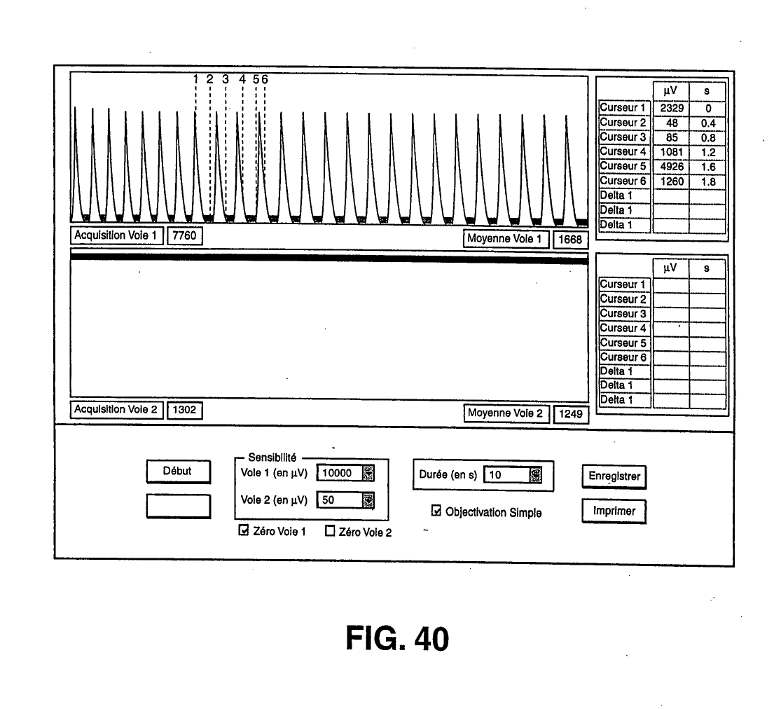

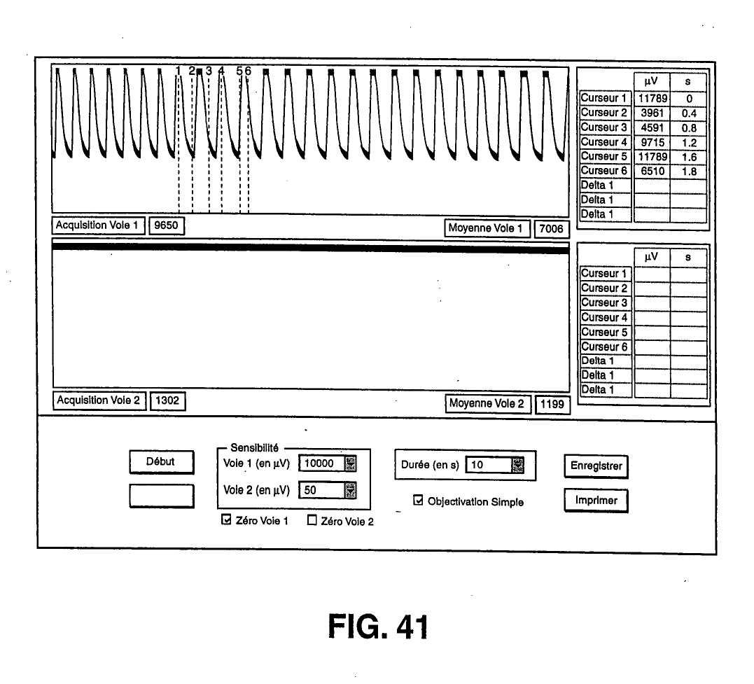

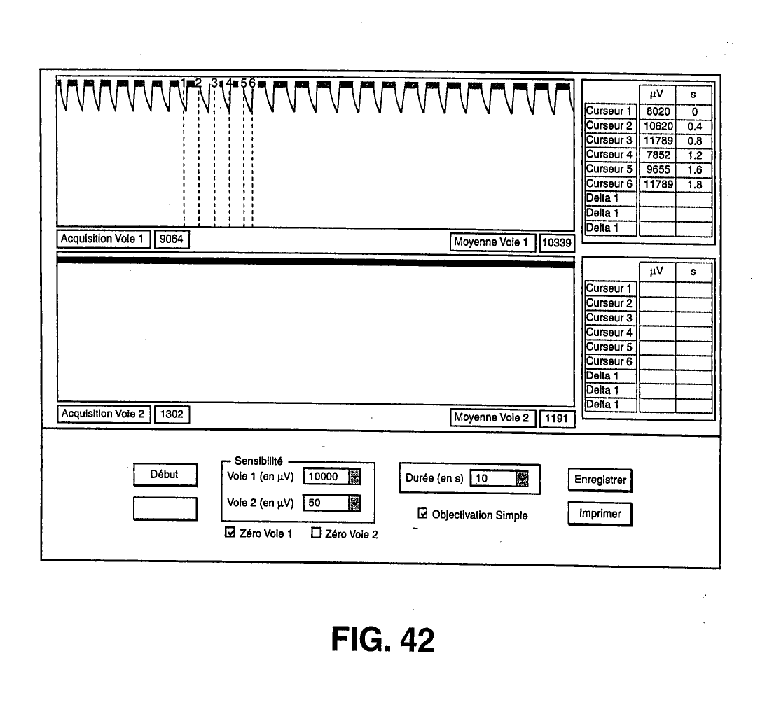

The following curves were obtained:

| | |
|---|---|
| Croco on lymphatic tissue (FIG. 38) | very mediocre result 909 |
| Needles in lymphatic tissue (FIG. 39) | average result 1729 |
| Croco on red muscle (FIG. 40) | average result 1668 |
| Needles in red muscle (FIG. 41) | good result 7006 |
| Croco on venous tissue (FIG. 42) | 10 339 very high activity |
| Needles in venous tissue (FIG. 43) | 10 893 very high activity |

Although the low quality of perivascular red muscles could distort certain results of this test, these multiple tests do show that the device exerts a very clearcut effect on the vein and a red muscle (in the manner of a vein pump, with a very pronounced contraction effect), more so than on the lymphatic system, where the effect is virtually zero.

This indicates that the square wave signals exert no significant effect on the lymphatic system.

Such tests also have the merit of validating all the other tests indicated above which have been performed, because they make it possible to duplicate the results announced by the manufacturer. It may be inferred that the tests performed with the FDLP and with the Datavein™ are significant.

The invention thus relates to a device for stimulation and treatment of the lymphatic system (this term applying for the sake of simplicity to all the effects and all the applications referred to in the foregoing), a device which comprises an electric pulse generator and at least two electrodes which are positioned on the epidermis of the subject to be treated, characterized in that such electric current pulses are not in the form of square wave signals.

In one specific embodiment the device is characterized in that the electric current pulses have a work time ranging from 1 to 12, preferably 1 to 8, preferably 2 or 6, millisec.

In one particular embodiment the device is characterized in that the electric current pulses have a rest time ranging from 300 to 900 millisec, preferably from 400 to 700 millisec, preferably 500 or 650 millisec.

In one specific embodiment the device is characterized in that the electric current pulses exhibit a current intensity of 6 to 300 μA, preferably 6 to 50 or 100 or 150 μA, or more, depending on the sensitivity (“feeling”) of the patient and on the number of electrodes (2, 4, 6, etc.; see below), as the expert will readily understand.

In another specific embodiment the device is characterized in that the electric current pulses exhibit a frequency of around 0.1 to 3 Hz, preferably around 1.99 Hz or around 1.52 Hz.

In another specific embodiment the device is characterized in that the electric current pulses exhibit pulse trains.

In another specific embodiment the device is characterized in that the electric current pulses preferably exhibit pulse trains of the 10/10 type.

In another specific embodiment the device is characterized in that the electric current pulses exhibit pulse trains with reversal of polarity between each sequence.

In one specific embodiment the device is characterized in that the electric current pulses are set at:

| Work time | 2 | millisec |
|---|---|---|
| Rest time | 500 | millisec |
| Pulse trains | 10/10 | |
| Frequency | 1.99 | Hz, |

this producing a “regulating” or “reeducating” current for treatment of pathological or deficient lymphatic systems.

In one specific embodiment the device is characterized in that the electric current pulses are set at:

| Work time | 6 | millisec |
|---|---|---|
| Rest time | 650 | millisec |
| Pulse trains | 10/10 | |
| Frequency | 1.52 | Hz, |

this producing a “stimulating” or “turbo” current applying a very strong effect of stimulating the lymphatic system. Consequently, this current will be more suitable for accelerating or activating the non-pathological lymphatic system.

In another specific embodiment the device is characterized in that the electric current pulses are applied by:

epidermal “contact” electrodes, of the flat type, carbonated or not, adhesive or not, with or without hydrogen, positioned on or adhering to the epidermis

and, optionally, in certain cases identified precisely by the practitioner

clips of the “alligator” type,

needles implanted in the cutaneous tissue.

In another particular embodiment the device is characterized in that the electric current pulses are applied by at least two electrodes.

In another particular embodiment the device is characterized in that the electric current pulses are applied by x electrode couples such as two (+)(−) electrodes or four (+)(−) electrodes covering two treatment areas, etc.

The applications of the invention, device and process, are represented by non-restrictive examples such as the following:

in the area of esthetics:

treatment of cellulitis

facial care (edema, undernourished skin)

scars

in the case of pathological conditions:

lymphostasis as described above

venous disorders

excessively strained muscles/muscular regeneration

scars

inflammation, tendinitis

lymphology/phlebology/traumatology/athletic traumatology/rheumatology/dermatology/surgery/geriatrics/gastroenterology/gynecology/obstetrics/treatment of burns in immunology

and, in the horse or other animals such as the dog in particular:

all pathological circulatory conditions, laminitis and other pathological excess stress conditions (muscular regeneration, scars, inflammation, tendinitis, tendinopathies including tendinitis, pathological muscular or osteo-articular conditions)

pathological condition of working muscles, in the horse,

and other analogous human or veterinary applications which will be obvious to and within the sphere of knowledge of the expert.

1. A device for stimulation of the lymphatic system in the human or animal, the horse in particular, characterized in that such device comprises a generator of electric pulses and at least two electrodes which are positioned on the epidermis of the subject to be treated so as to cause electric stimulation trains to pass into the human or animal body involved (hereinafter referred to jointly and separately as the “body”), each stimulation or pulse being characterized in that
square wave signals are not involved
the excitation period is shorter than the rest or relaxation period;
the excitation period ranges from 2 to 8 milliseconds;
the rest or relaxation period ranges from 400 to 850 milliseconds.

2. The device as specified in claim 1, wherein the intensity of the electric current applied is lower than or equal to 1 mA (one milliampere), and is preferably of the order of 6 to 300 μA (one microampere), preferably 6 to 50 or 100 or 150 or more, depending on the number of electrodes.

3. The device as specified in claim 1, wherein such device is suitable for application of trains of pulses or “bursts” of 5/5 to 10/10 or to 15/15, preferably 10/10.

4. The device as specified in claim 1, wherein each train of pulses exhibits reversal of polarity relative to the preceding train.

5. The device as specified in claim 1, wherein the frequency of each pulse ranges from 0.1 to 3 Hertz, and preferably ranges from 0.7 to 2.5 Hertz, preferably near 11.5 or 2 Hertz.

6. The device as specified in claim 1, wherein the pulses of electric current exhibit a working period ranging from 1 to 12, preferably 1 to 8, milliseconds, preferably 2 or 6 milliseconds.

7. The device as specified in claim 1, wherein the pulses of electric current exhibit a rest period ranging from 300 to 900 milliseconds, preferably 400 to 700 milliseconds, preferably 500 or 650 milliseconds.

8. The device as specified in claim 1, wherein such device is suitable for delivery of:
a first type of current which is a “regulating or reeducating” current, useful primarily for treatment of pathological conditions of the lymphatic system and centered on a setting of around
2 milliseconds of work, 500 milliseconds of rest, 10/10 sequences >>1.99 Hz and/or
a second type of “stimulating” or “turbo” current which provokes stimulation of the lymphatic system much stronger than the preceding one, useful primarily in other applications of the device, physiological labor, and centered on a setting of around:
6 milliseconds of work, 650 milliseconds of rest, 10/10 sequences, >>1.52 Hertz.

9. The device as specified in claim 1, wherein the pulses of electric current exhibit preferably pulse trains of the 10/10 type.

10. The device as specified in claim 1, wherein such device comprises at least two “contact” electrodes of the flat type, carbonated or non-carbonated, adhesive or non-adhesive, with or without hydrogel, positioned on or adhering to the epidermis or
“alligator” clips
needles implanted in the cutaneous tissue.

11. The device as specified in claim 1, wherein such device comprises x couples of (+)(−) electrodes, such as two (+) (−) electrodes or four (+)(−) and (+)(−) electrodes covering two areas of treatment, etc.
